(19) United States
(12) Patent Application Publication
(10) Pub. No.: US 2013/0085394 A1
(43) Pub. Date: Apr. 4, 2013

(54) GLOVE WITH INTEGRATED SENSOR

(71) Applicant: Sonivate Medical, Inc., Beaverton, OR (US)

(72) Inventors: Scott S. Corbett, III, Portland, OR (US); Ronald W. Schutz, Portland, OR (US)

(73) Assignee: Sonivate Medical, Inc., Beaverton, OR (US)

(21) Appl. No.: 13/645,317

(22) Filed: Oct. 4, 2012

Related U.S. Application Data
(60) Provisional application No. 61/543,228, filed on Oct. 4, 2011.

(51) Int. Cl. A61B 8/12; A61B 8/00

(52) U.S. Cl. CPC A61B 8/12; A61B 8/4422; A61B 8/4488
USPC 600/462

(57) ABSTRACT

A sensing apparatus which comprises a glove having an integrated sensor and electrical conductive structure which are at least partially embedded in the material of the glove, the material of the glove accommodating or enhancing the function of the sensor.

### CROSS-REFERENCE TO RELATED APPLICATIONS

This application claims the benefit of U.S. Provisional Application No. 61/543,228, filed on Oct. 4, 2011, the entire disclosure of which is hereby incorporated herein by reference.

### BACKGROUND OF THE INVENTION

As the medical use of ultrasound probes has become increasingly pervasive, the challenge of protecting patients from pathogens born on such probes has become increasingly pressing. Ultrasound probes are used in body cavities and in procedures where skin or mucus membranes are penetrated, where they can easily spread infectious disease. Ultrasound probes that are used for endocavity examinations require high level disinfection, and a probe that is used during a procedure where skin or mucus membranes are penetrated must be sterile. However, it is very difficult or even impossible to sterilize or even accomplish high level disinfection of most ultrasound probes. Ultrasound probes typically cannot be sterilized in an autoclave. Many disinfection agents such as isopropanol are not high level disinfectants when used as a wipe, and equipment must be soaked in such an agent in order to be properly disinfected. Most manufacturers of ultrasound probes recommend that they not be soaked.

To overcome these challenges, in standard practice a disposable commercial probe cover is used to prevent contact between probe and patient. However, these probe covers present numerous problems with any ultrasound procedure. The probe covers may create artifacts or acoustic distortions, especially if they create air pockets between the probe and the patient. They can be cumbersome, and can slip. Moreover, high rates of perforation (8-81%) have been found in studies tracking leakage rates of commercial probe covers.

Finger mounted ultrasound probes present numerous advantages such as their ability to be inserted into small spaces with minimal patient discomfort and better ergonomics for medical personnel. However, the disadvantages of the standard method of ensuring probe sterility, a probe cover, are compounded when the probe is a finger probe. One could put a probe cover over a finger probe. However, an ultrasound probe cover will not fit a probe and its cable snugly. A loose fitting probe cover may create only minimal difficulties with a conventional probe, but will be problematic when used with a finger probe. A standard probe cover which accommodates the mass of the probe will be too big for the finger. The excess material will interfere with tactile sensation, and could be very cumbersome, especially when the operator needs to use the finger in question for anything else, such as operating another instrument, palpating anatomy, or tying a suture. If the probe cover ends at the base of the finger, the cable which connects the probe to other system components such as a processor will not be covered and will present an unacceptable infection risk. A surgical glove covers the entire hand; however, a conventional surgical glove presents challenges when placed over the hand and the probe. Such a glove sized appropriately for a human finger alone cannot be squeezed over an ultrasound probe without straining the material and giving rise to a concern that the strain might weaken the glove, potentially causing leaking and infection risk.

Moreover, the presence of even tiny pockets of air in the tip of the glove can interfere with the ultrasound image. Air pockets trapped between the finger of the glove and the ultrasound probe are acoustically opaque and will create distortions or artifacts which render the ultrasound image unreliable. Tiny amounts of air in the tip of a glove would be nearly impossible to eliminate once the glove is in place on the hand. Lubricant such as ultrasound gel is typically used to displace air, but gel cannot simply be squeezed into the finger tip of a standard, sterile surgical glove without extensive manipulation and likely destruction of sterility, and once the glove is on the hand, it is impossible to add more gel without removing the glove.

While some prior art references make passing reference to placing a glove over a finger mounted probe, they do not address the problems associated with actually doing so. These problems remain unresolved.

A sensor typically connects to other system components such as displays or processors using a coaxial cable, which is strong enough to stand up to regular manipulation without being damaged. However, when a round cable is pressed between a user's hand and a glove, it is uncomfortable and compromises the capability of the hand. Flex circuits can be flat, smooth, and flexible, and can more easily be situated within a glove without user discomfort or compromise of the glove. However, bare flex circuits are more fragile than coaxial cable, and cannot be sterilized.

What is needed is a finger mounted sensor such as an ultrasound transducer which does not present infection risk when used intracavity and which does not interfere with the other activities of a user.

### SUMMARY OF THE INVENTION

A sensing apparatus, comprising a glove adapted to be worn on the hand of a user, at least a section of said glove having an inner layer of material and an outer layer of material disposed substantially adjacent to said inner layer, said inner and outer layers of material thereby defining a region in between said inner and outer layers; a transducer being at least partially disposed in said region; and at least one first signal propagator having first and second ends, said first end being in electrical communication with said transducer and said second end being disposed for allowing electrical communication between the transducer and a second signal propagator.

The foregoing and other objectives, features, and advantages of the invention will be more readily understood upon consideration of the following detailed description of the invention taken in conjunction with the accompanying drawings.

### BRIEF DESCRIPTION OF THE SEVERAL DRAWINGS

FIG. 1 is a top view of the dorsal aspect of a glove with integrated sensor as described herein.

FIG. 2 is a top view of the palmar aspect of a glove with integrated sensor as described herein.

FIG. 3 is a cross sectional side view of the one finger of the glove with integrated sensor as described herein as mounted on a user's finger.

FIG. 4 is a cross sectional view of one embodiment of the glove with integrated sensor described herein.

FIG. 5 is a cross sectional view of one embodiment of the glove with integrated sensor described herein.

FIG. 6 is a perspective view of a work piece representing a step in a manufacturing process for one embodiment of the glove with integrated sensor and connective structure described herein.

FIG. 7 is a perspective view of a work piece representing a step in a manufacturing process for one embodiment of the glove with integrated sensor and connective structure described herein.

FIG. 8 is a perspective view of a work piece representing a step in a manufacturing process for one embodiment of the glove with integrated sensor and connective structure described herein.

FIG. 9 is a perspective view of a work piece representing a step in a manufacturing process for one embodiment of the glove with integrated sensor and connective structure described herein.

FIG. 10 is a perspective view of a work piece representing a step in a manufacturing process for one embodiment of the glove with integrated sensor and connective structure described herein.

FIG. 11 is a perspective view of a work piece representing a step in a manufacturing process for one embodiment of the glove with integrated sensor and connective structure described herein.

FIG. 12 is a perspective view of a work piece representing a step in a manufacturing process for one embodiment of the glove with integrated sensor and connective structure described herein.

FIG. 13 is a cross sectional view of one embodiment of the glove with integrated sensor described herein.

### DETAILED DESCRIPTION OF PREFERRED EMBODIMENTS

Disclosed herein is a glove with an integrated sensor such as an ultrasound transducer and attached connective structure. Such an ultrasound glove 6 could be a sterile, single use item, and therefore could be safely used for use in body cavities or in surgery without the need to withstand repeated sterilization. In order to be suitable for integration into a glove, an ultrasound transducer and an interconnect structure that connects the transducer with other system components must be small, light, and relatively flat so that they do not unduly interfere with the function of the glove or the hand within the glove. The material of the glove must permit and even facilitate the function of the ultrasound transducer.

A glove suitable for this purpose should be suitable for use as an examination or surgical glove. It should be made of a vapor and moisture resistant material such as latex, vinyl, nitrile rubber, or neoprene which provides a barrier to bodily fluids and contaminants such as pathogens.

An ultrasound transducer suitable for use as described herein may be formed from an array of piezoelectric elements, often made from a ceramic material such as lead zirconate titanate. A transducer made with such an array often requires one or more layers of backing material, matching material, and a lens.

Alternately, a suitable transducer may be made from a MEMS sensor such as a CMUT sensor, or from a flex circuit. A MEMS sensor is a low cost ultrasound transducer that can emit and receive ultrasound signals. Examples include CMUT sensors which consist of vibrating silicon diaphragms produced using semiconductor fabrication techniques and filled with conductive materials. The sensor has a interconnect pattern on the bottom of the chip, which is readily terminated onto a similar pattern on flex circuit. Alternatively, the sensor itself can be made from the flex circuit, by laser patterning the diaphragm from the flex itself. The reduced stiffness of the flexible material increases the bandwidth of the sensor over a stiffer silicon diaphragm. Additionally, such a sensor may reduce or eliminate the need for matching layers. The sensor array can be a linear, phased, 1.5D or 2D array to form various scanning beams.

A high performance acoustically absorptive backing material 10 may be affixed behind the sensor array 12, so that the elements of the interconnect structure 8 connected to piezoelectric elements are encapsulated between backing material 10 and the sensor array 12. Backing is employed to reduce the duration and spatial length of pulses of ultrasonic energy emitted by the sensor elements in response to voltage. This attenuation of ultrasonic energy reduces artifacts and increases resolution in the resulting images. Backing material 10 may be as disclosed in U.S. Pat. No. 4,779,244, issued Oct. 18, 1988, which is incorporated herein by reference as if fully set forth herein. In that patent a backing material having an acoustic absorbance equal to or greater than 60 db/MHz/cm is disclosed. By way of illustration, using such a material and given the need to attenuate a typical 5 MHz ultrasound signal emitted from the back of the array by approximately 150 dB, over a two-way trip through the backing material (so as not to interfere with a desired ultrasound image), the array backing would need to have a thickness of approximately 150 dB÷[(2)(5 Mhz) (60 dB/MHz/cm)]=2.5 mm, which allows a very low profile for a transducer intended to fit on the finger. Different backing materials having different characteristics may be used instead.

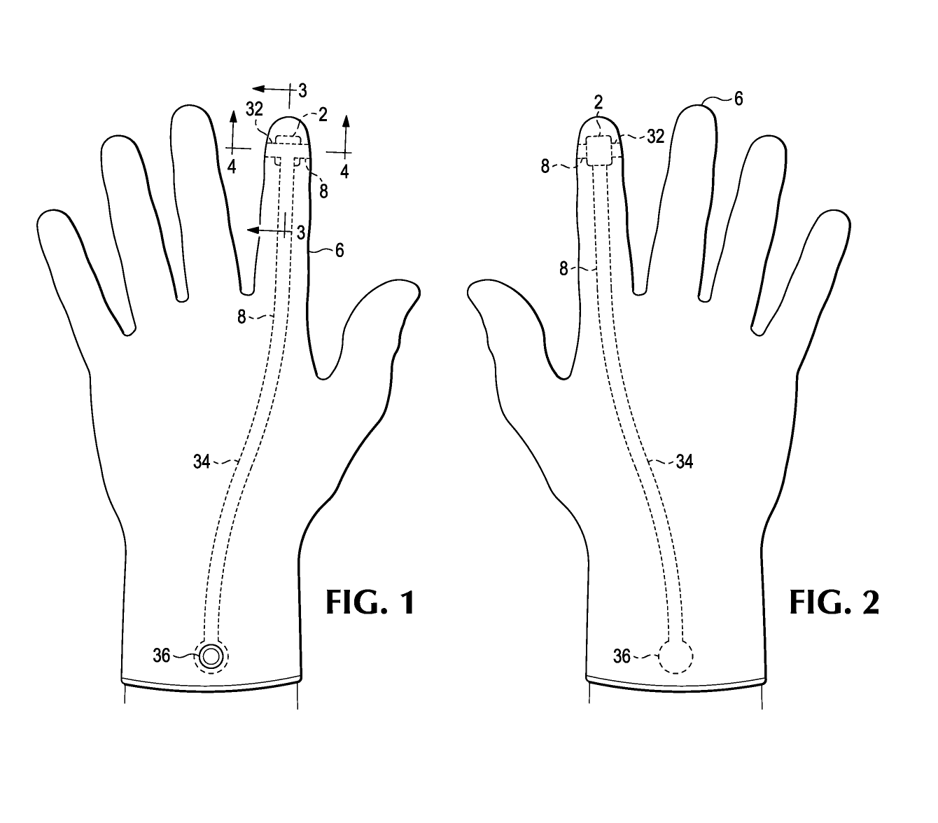

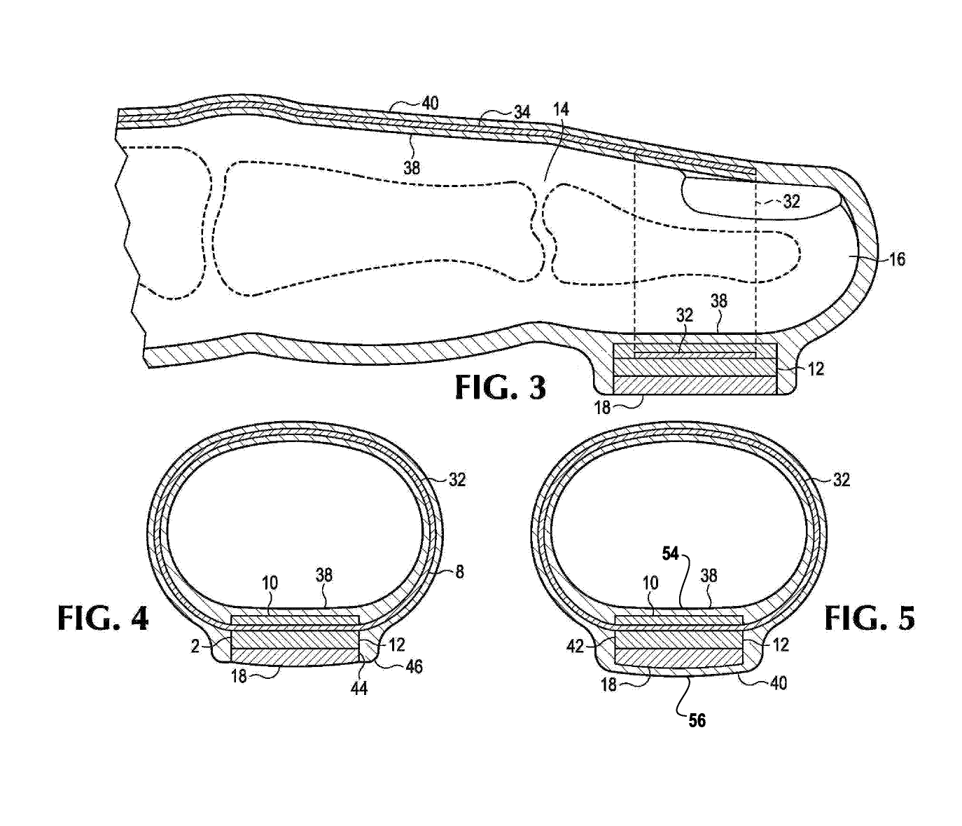

Depending on the nature of the sensor array used, a fairly rigid component should be placed behind the sensor array. This component may be a backing layer or a region of the interconnect structure directly behind the sensor array. A rigid component in this location helps ensure that the sensor itself does not flex during use. Flexing of the sensor array may interfere with the beam forming and sensing operation of the array, which are affected by geometric stability. An array and associated rigid structure embedded in a glove worn by a user should not extend over the user's distal interphlangeal joint 14 so that it will not impede bending of the user's finger 16. As shown in FIGS. 1 and 3, the sensor and signal propagators such as flex circuit interconnected with the sensor should be oriented on the glove so as to minimize impact on the use of a hand that wears it. The distance 4 between the fingertip and the sensor should be relatively short, and the sensor and any associated signal propagator should be more or less centered on the finger such that the distance 3 between the center of the structure and each edge of the finger is substantially equal.

A lens 18, made from a substance which has an appropriate impedance and sound velocity, completes the sensor assembly. The lens 18 may focus the beam emitted by the array, or in the case of an array that does not need focusing, it may simply prevent contact between the array and the surrounding environment.

The sensor assembly must be connectable to external components, such as processors or monitors, with an interconnect structure 8. This interconnect structure 8 must be suitable for integration into a glove, in one embodiment through encapsulation between layers of material in the glove. Ideally, it will be flexible and as small and/or as thin as possible. Moreover, it should be situated within the glove material in such a way as to minimize interference with the function of a user's hand.

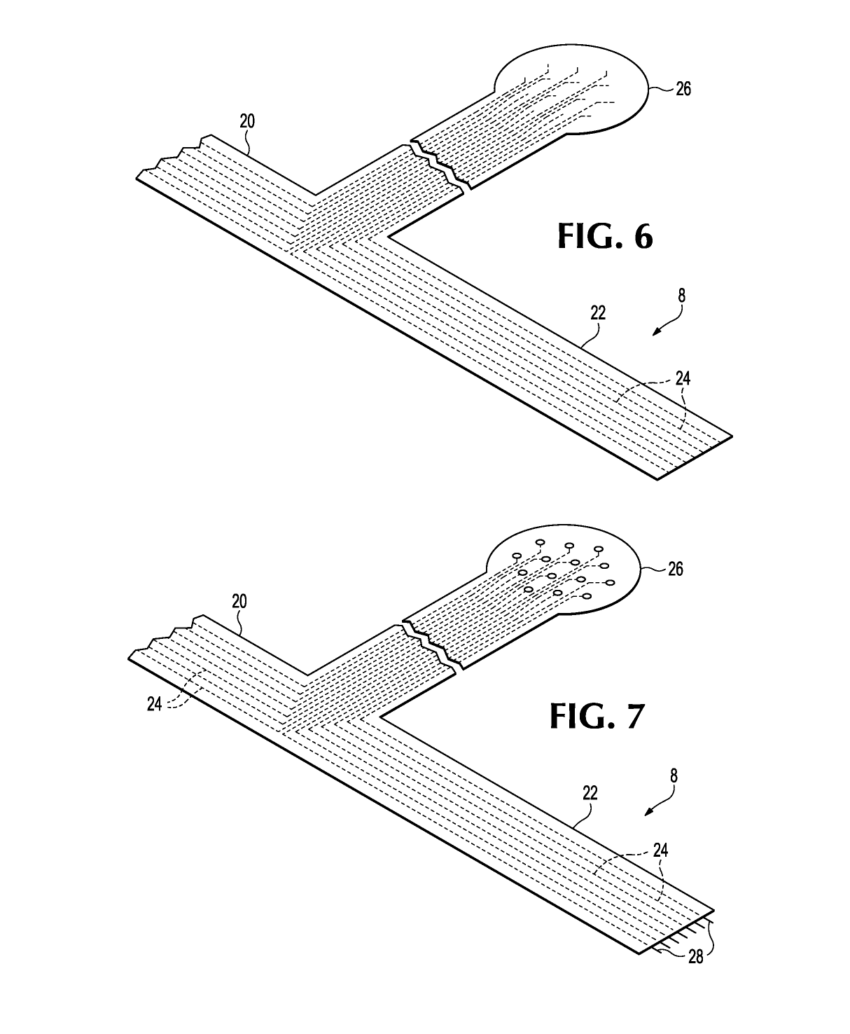

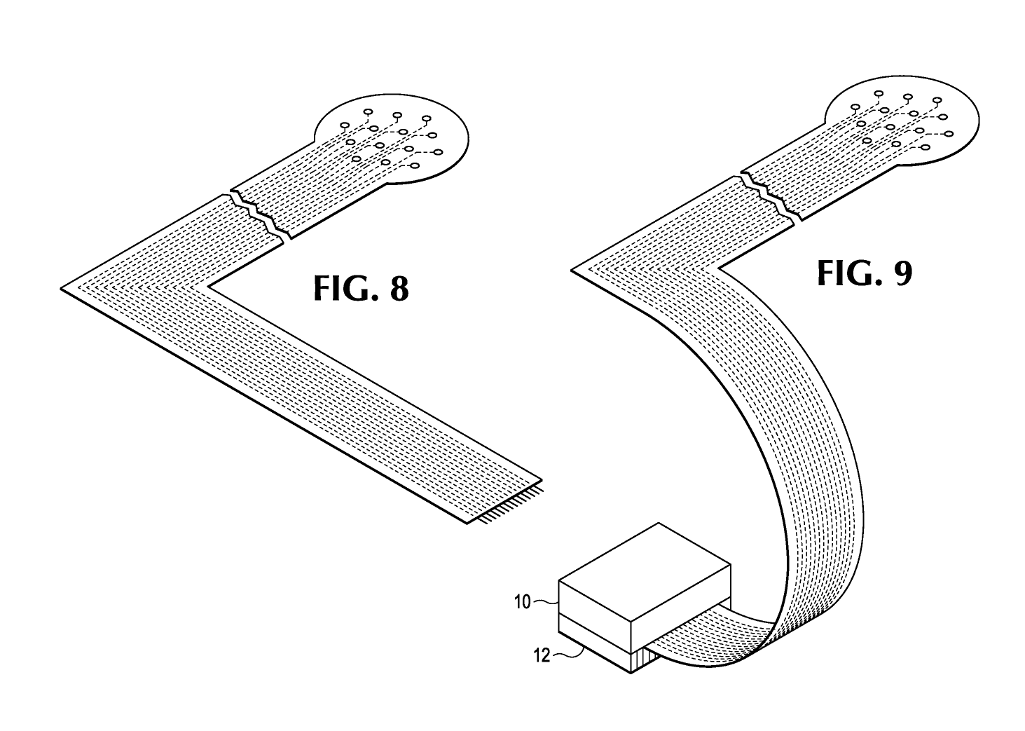

Such an interconnect structure should be operably connected to the sensor array, which emits from the palmar aspect of the glove, and should provide connectivity to other components on the dorsal side of the glove, where it is out of the user's way. Referring to FIGS. 6-7, which form a part of this disclosure, in one embodiment construction of a transducer with such an interconnect structure 8 may begin with the creation of a T-shaped piece of flex circuit. Alternatively, two or more L-shaped pieces may be overlapped or placed side-to-side to form a T shape. The distal end T-shaped top bar includes a first branch 20 and a second branch 22. Each of several conductive traces 24 turns at the T-junction and extends from proximal end 26 to the end of either branch 20 or 22. While for illustrative clarity only 7 conductive traces 24 are shown in each branch 20 or 22, a larger number, such as 32 separate parallel traces 24, may be included in a layer of the flex circuit, and more than one layer, for example 4 layers or as many as 8 layers, may be included. A set of bare trace ends 28 are formed at the free ends of branches 20 and 22 by removing the end of the plastic of flex circuit from about traces 24, typically by laser ablation. Each of several flex circuit layers may typically have a thickness of only 0.3 mm, so a cable of 8 flex circuit layers can still be conveniently flexible and have a thickness of no more than about 2.5 mm. The ribbon-like cable may have a width, depending on the number and size of the traces, in the range of 1-2 cm.

An ultrasound transducer 2 may be formed by connecting the trace ends 28 to respective transducer elements such as pieces of piezoelectric material arrayed alongside one another. The trace ends 28 may be interdigitated and connected to alternately located elements from the two sides of the transducer 2. The elements of piezoelectric material of the ultrasound transducer may be arrayed with each transducer element being connected to a unique trace and to a common ground plane bus. In one contemplated embodiment a conveniently located set of ultrasound elements may be connected to trace ends of one branch 20, while another set of transducer elements are connected to trace ends of the other branch 24.

The bare trace ends are interconnected with a CMUT sensor in much the same way by virtue of an area interconnect scheme on the dorsal aspect of the sensor. That interconnect scheme may include channels cut through the sensor wafer and into the highly conductive silicon substrate which isolate the elements and create silicon pillars that form signal electrodes that can be electrically interconnected with the traces. Alternatively, an interposer may be used on the back of the chip.

Referring to FIGS. 4, 5, 10 and 13, once connected to the sensor, the branches 20 and 22 may be flexed to form a ring 32 that can fit about a user's finger, so that ultrasound transducer array faces downwardly and emits from the palmar aspect of the glove, and the cable or flex circuit which connects the transducer with the rest of the imaging system is routed along the dorsal aspect of the user's hand where it is out of the way.

A structure which connects with the sensor on the palmar aspect of the glove and connects with other components of the ultrasound system on the dorsal aspect may take a number of different configurations. L-shaped pieces of flex circuit could be used, rather than a single T-shaped piece, which would permit the step of connecting bare traces 28 to piezoelectric transducer elements to be performed with the L-shaped pieces of flex circuit lying flat, thereby greatly easing this connective task. The lateral branches of L-shaped pieces may then be curled up and the longitudinal portions may be interleaved and overlapped at the top, thereby forming an annulus that fits about the finger at the end of a multi-layer flex circuit cable.

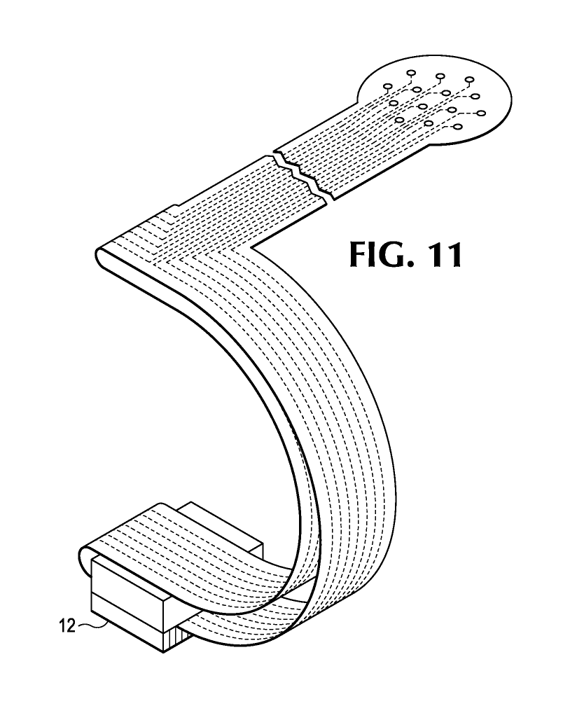

The branches extend around the finger to terminate in a junction with more flex circuit or other conductive material 34 on the dorsal aspect of the user's hand so that it does not interfere with the function of the hand. The conductive material 34 terminates in a connector 36 at the end of the glove. As shown in FIGS. 9 and 11, a single sided connective structure can be created, for example by laying one branch against another. Alternatively, the transducer may be operatively connected to a single piece of flex circuit. That flex circuit could extend around one side of a finger, or it could extend from the array at the tip of the finger, run along the palmar surface of the finger, and then wrap around to the dorsal surface of the hand so that it emerged at the end of the glove in the vicinity of the back of the wrist. Any preferably flat interconnect may be used instead of flex circuit.

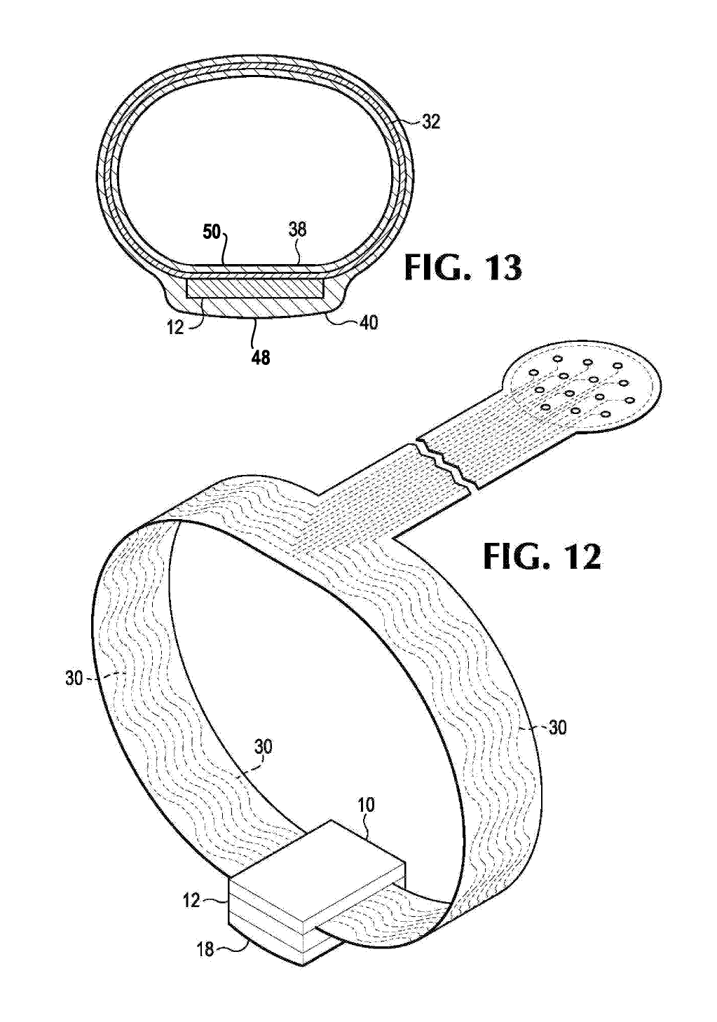

The branches of the flexible interconnect structure or the entire structure can be made from different dielectrics so as to make more practical the inclusion of the sensor and interconnect structure into the glove. For example, as shown in FIG. 12, the branches 20 and 24, which form the portion of the interconnect structure that wraps around the finger, can be made of a more flexible silicone material so as to accommodate stretching during use. The traces 30 on the substrate can also be formed in a serpentine or “wavy” fashion so as to accommodate a certain amount dimensional change such as stretching in response to stress without breaking. Glove material should also be resilient and able to stretch to accommodate the insertion and movement of a user's hand.

The transducer elements may be arranged and oriented transverse to the direction of the finger so as to create an image slice in the same direction (longitudinal to) the finger. In an alternative embodiment, the transducer elements may be oriented in the same direction as the finger, so that an image slice is formed transverse to the finger. Such an orientation would require the branches of flex to be folded so that they can be connected to elements which are longitudinal to the finger and yet can and extend around the sides of the finger to the dorsal aspect. An alternative embodiment uses elements arranged in both orientations to create a bi-plane probe capable of creating scan planes both parallel and transverse to the finger orientation. In such an embodiment, two different arrays of elements would be arranged in a T configuration or an inverted T configuration on the finger, and could each be connected to its own annulus of flex circuit. The two arrays may both be placed on the finger distal to the user's interphlangeal joint 14, or one array may be on either side of the joint, allowing the glove (and consequently the finger) to flex at the joint.

A transducer has an inward facing aspect 54 and an outward facing aspect 56. A glove having two layers of material has an inner layer 38 which is proximal to the inward facing aspect 54 of the transducer 2, and an outer layer 40 which is proximal to the outward facing aspect 56 of the transducer. The transducer, including sensor array 12 and interconnect structure 8, and optionally including a backing 10 and lens 18, can be incorporated into a glove in a variety of ways. As much as the transducer and interconnect structure as possible may be encased between inner 38 and outer 40 layers of glove material so that the encased components are isolated from the patient.

The components can be sandwiched between two layers of glove material in a variety of manners. For example, a mold can be dipped into glove material then cured, the transducer 2 and interconnect structure 8 can be placed over the mold, and then the mold can be dipped again. Alternatively, limited portions of the glove such as the finger bearing the sensor may be double-dipped in this manner.

Alternatively, the transducer 2 and the interconnect structure 8 can be embedded into one or more specially formed pockets 42 of glove material, so that the glove material encases or partially encases the transducer and the interconnect structure. The pockets can be sealed after the transducer and interconnect structure are placed within them.

As shown in FIG. 4, the transducer 2 may be partially encapsulated or embedded in the glove so that it is integral with the glove but not necessarily fully encapsulated. The transducer may be attached to the outside of the glove or within a cavity 44 formed in the glove with the glove material 46 forming a seal around the lens 18 and the flex circuit interconnect structure 8 affixed to the glove or embedded in the glove. Alternatively, the transducer may reside on both sides of a layer of glove material 46. For example, the transducer array and/or backing material may reside on the inside of a layer of glove material, then matching layers and/or a lens may be affixed to the outside of the glove. The transducer and interconnect can also be adhered or affixed to the inner surface of a sterile glove or glove layer, with or without additional glove or glove layers.

The glove or areas of the glove surrounding the sensor may be made of materials which are acoustically transparent, such as urethane, polyvinyl alcohol or other materials that have a low acoustic attenuation and acoustic impedance similar to that of a human body. Such materials can act as a lens. If such materials were used in the outer layer of glove encasing the sensor, a separate lens component could be omitted, and that glove layer 48 could act as a lens. Silicone rubber, modified silicone rubber, or a room temperature vulcanizing polymer may be used for this purpose. The glove material facing the transducer array should be acoustically transparent, but may also have a lower velocity of sound than the human body such that it is acoustically refractive and can focus the acoustic beam in the relevant elevation plane. Lenses for ultrasound probes are frequently made form a two-part silicone room temperature vulcanizing rubber material. A rubbery material such as that could be used to make a glove or part of a glove, and could be bonded or adhered to other rubbery materials making up other parts of a glove.

The area of glove 50 behind the piezoelectric elements, between the elements and the surgeon's hand, can be made of an acoustically absorptive material which functions as a backing. Rubbery materials appropriate for gloves with the addition of substances such as titanium dioxide or nanopowder such as ceramic powder, or such as an epoxy filled with rubber particles which have small micro metallic scatters in them, are appropriate for this purpose. If such materials are incorporated into the glove, no separate backing may be needed. A backing material may not be used at all with some sensors.

A section of glove made of a material having an impedance value between that of the transducer element and human tissue could replace one or more matching layers in the ultrasound transducer. Alternatively, glove material may function as a radio frequency interface shield. Such a shield may be made of a polymer sputter coated with a thin metal such as gold. The relevant glove material may be treated so that it is capable of acting as such a shield.

If the glove material is to function as a component of the transducer, it must be appropriately located. For example, glove material which functions as backing 50 must be distal to the sensor array 12, or proximate to the inward facing aspect of the transducer 2. Glove material which functions as a lens 48 or as a matching layer must be located between the sensor array and the surface to be scanned, proximal to the outward facing aspect of the transducer.

A housing with a lens may cover the acoustic array. The outer glove layer may extend over the lens, or the outer glove layer may have an opening corresponding to the lens. The opening should be leak proof, and can utilize a leak-proof molded seal in order to maintain the glove's structural integrity, or the glove material may be adhered to the lens with epoxy or polymeric adhesive.

A glove with embedded sensor may be made through additive manufacturing processes, which would permit the creation of a seamless glove with different areas made of different substances or having different characteristics. Additive manufacturing could also be employed to create a glove which seamlessly encapsulates sensor components and connective structural components.

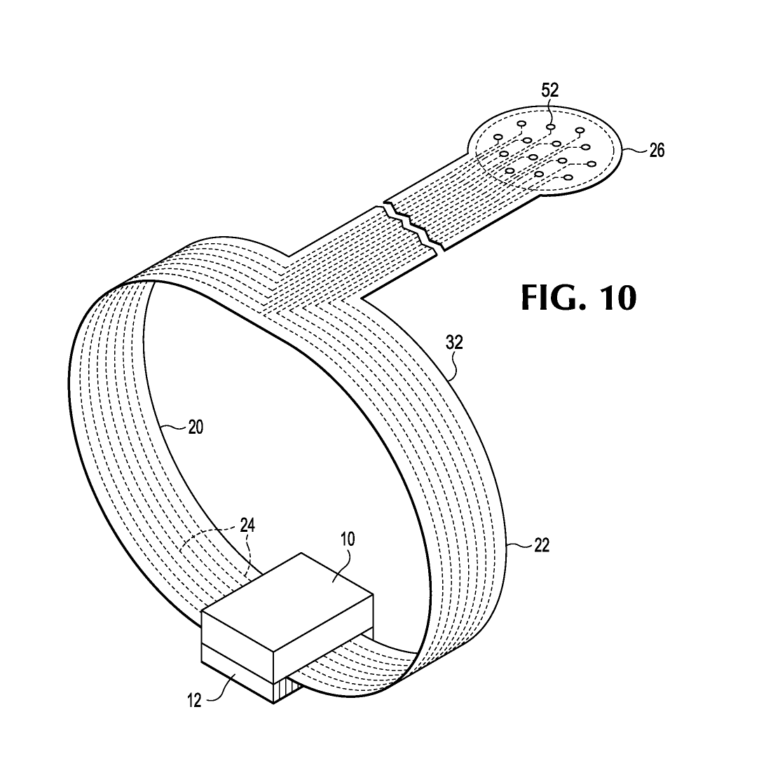

The ultrasound transducer must operably connect to other elements or components of the ultrasound systems, such as a processor/monitor, which may be one unit or more, so that images can be generated and displayed. This connection may occur wirelessly. The proximal end of the flex circuit may protrude from the glove with a circuit pattern of connector tabs contained thereon which can serve as a connector 36. A flex tip can be inserted into a simple flex based connector which has opposing pads matching the pattern of connector tabs on the flex, to make a connection. Referring to FIG. 10, at the proximal end of the flex circuit 26, a set of electrical contact points 52 are formed by removing the flex circuit plastic down to each trace 24, in a particular spot. Conductive material may be deposited onto contacts 52, so that they are not recessed. Alternatively, a surface coating material covers flex circuit conductive traces 24 so that only connector contacts 52 are left exposed on the surface of flex circuit. Connections to the flex traces may be formed by laser or mechanical drilling and subsequent plating to form a monolithic integrated connector assembly.

The flex circuit may be embedded in the glove material or between glove layers for the length of the glove, emerging at the end of the glove. Alternatively, the flex circuit may emerge through an opening in the outer glove layer, perhaps in the vicinity of the back of the hand. Such an opening should be surrounded by a leak-proof molded seal which would protect the glove's integrity.

The transducer array may also be in electronic communication with a wireless transmitter which may transmit information to a processor to convert it into an image. The transmitter may include a receiver such that control signals may be transmitted to the transducer array wirelessly. These control signals may perform functions such as changing the operating frequency of the array. This transmitter/receiver may make the transducer wireless and eliminate the need for a wired connection to a processor. Such a transducer will still need a power source, and can be connected to a power cell or batteries located on the glove, on the user's clothing, or attached to the user, perhaps mounted on the user's arm or wrist. Flex circuit or other planar interconnect may be used to connect the transducer to the power source.

The glove can also include active electronic components which perform beam forming or signal conditioning functions. Silicon die containing active electronics can be thinned to the point that the structure is low profile and flexible allowing integration into a glove. Signal conditioning and beam forming functions reduce the bandwidth required for wireless signal transmission. Ideally the signals from the array elements are summed and processed as close to the sensor as possible. The integrated circuit can reside directly behind the active sensor region of the array or could reside further back in the body of the glove.

Any sort of signal propagator can be used to electrically interconnect the transducer and other components of an imaging system. For example, connections between the transducer and the processor may be optical instead of electrical. Alternatively, power may be provided to the transducer through induction, or through microwaves, which would permit the wireless provision of energy. Especially when elements of the ultrasound system such as a power source or electrical interconnect are located on the surgeon's gown, the gown and the glove may be magnetically coupled so as to assist in maintaining connectivity between the ultrasound transducer and the elements coupled to the gown. All elements permanently connected to or affixed to surgical gowns must be either disposable or sterilizable. Elements which can be removed from the gown may be separately sterilized or disinfected then reattached to the gown, or may be provided as pre-sterilized, single use items.

The terms and expressions which have been employed in the foregoing specification are used therein as terms of description and not of limitation, and there is no intention in the use of such terms and expressions of excluding equivalents of the features shown and described or portions thereof, it being recognized that the scope of the invention is defined and limited only by the claims which follow.

What is claimed is:

1. A sensing apparatus, comprising:
a glove adapted to be worn on the hand of a user, at least a section of said glove having an inner layer of material and an outer layer of material disposed substantially adjacent to said inner layer, said inner and outer layers of material thereby defining a region in between said inner and outer layers;
a transducer being at least partially disposed in said region; and
at least one first electrical conductor element having first and second ends, said first end being in electrical communication with said transducer and said second end being disposed for allowing electrical communication between the transducer and a second electrical conductor element.

2. The sensing apparatus of claim 1 wherein said transducer is an ultrasound transducer.

3. The sensing apparatus of claim 1 wherein said electrical conductor comprises flex circuit.

4. The sensing apparatus of claim 1 wherein said inner layer of material is adapted to attenuate ultrasonic energy.

5. The sensing apparatus of claim 1 wherein said inner layer of material and said outer layer of material each has an acoustic absorbance value, and said acoustic absorbance value of said inner layer is higher than said acoustic absorbance value of said outer layer.

6. The sensing apparatus of claim 3 wherein said flex circuit is at least partially located in said region between said inner layer and said outer layer.

7. The sensing apparatus of claim 1 wherein at least a portion of said outer layer of material is acoustically transparent.

8. The sensing apparatus of claim 6 wherein said transducer comprises a sensor array, said sensor array having a first acoustic impedance value and at least a portion of said outer layer of material having a second acoustic impedance value, said second acoustic impedance value being lower than that of said first acoustic impedance value.

9. The sensing apparatus of claim 2 wherein said glove is adapted to be worn on a user's hand such that said transducer is located on a user's finger, and said transducer comprises elements which are disposed to be arranged transversely to an axis of a user's finger when said glove is worn.

10. The sensing apparatus of claim 2 wherein said glove is adapted to be worn on a user's hand such that said transducer is located on a user's finger, and said transducer comprises elements which disposed to be arranged perpendicularly to an axis of a user's finger when said glove is worn.

11. A sensing apparatus, comprising:
a glove adapted to be worn on the hand of a user, at least a section of said glove having an inner layer of material and an outer layer of material disposed substantially adjacent to said inner layer, said inner and outer layers of material thereby defining a region in between said inner and outer layers;
a transducer being at least partially disposed in said region;
at least a first signal propagator having first and second ends, said first end being in electrical communication with said transducer and said second end being disposed for allowing electrical communication between the transducer and a second signal propagator, at least one said signal propagator being at least partially disposed in said region.

12. The sensing apparatus of claim 11 wherein both said at least one signal propagator at least partially disposed in said region and said inner and outer layers substantially adjacent to said signal propagator are adapted to be capable of dimensional expansion in response to stress exerted by said hand of said user.

13. The sensing apparatus of claim 11 wherein said transducer is an ultrasound transducer.

14. The sensing apparatus of claim 11 wherein said signal propagator comprises flex circuit.

15. The sensing apparatus of claim 11 wherein said inner layer of material is adapted to attenuate ultrasonic energy.

16. The sensing apparatus of claim 11 wherein at least a portion of said inner layer of material substantially adjacent to said sensor and at least a portion of said outer layer of material substantially adjacent to said sensor have an acoustic absorbance value, and said acoustic absorbance value of said inner layer is higher than said acoustic absorbance value of said outer layer.

17. The sensing apparatus of claim 11 wherein at least a portion of said outer layer of material is acoustically transparent.

18. The sensing apparatus of claim 11 wherein said transducer comprises a sensor array, said sensor array having a first acoustic impedance value and at least a portion of said outer layer of material having a second acoustic impedance value, said second acoustic impedance value being lower than that of said first acoustic impedance value.

19. The sensing apparatus of claim 13 wherein said glove is adapted to be worn on a user's hand such that said transducer is located on a user's finger, and said transducer comprises elements which are disposed to be arranged transversely to an axis of a user's finger when said glove is worn.

20. The sensing apparatus of claim 13 wherein said glove is adapted to be worn on a user's hand such that said transducer is located on a user's finger, and said transducer comprises elements which disposed to be arranged perpendicularly to an axis of a user's finger when said glove is worn.
